(19) United States
(12) Patent Application Publication
(10) Pub. No.: US 2011/0302734 A1
(43) Pub. Date: Dec. 15, 2011

(54) ITEM FOR CLEANING THE SKIN

(75) Inventors: Anne-Gaelle Clermont, Colmar (FR); Philippe Gregoire, Les Andelys (FR); Bernard Louis Dit Picard, Amfreville La Campagne (FR); Bruno Bret, Wintzenheim (FR); Alexandre Da Silva, Neuf Brisach (FR)

(73) Assignee: GEORGIA-PACIFIC FRANCE, Bois-Colombes (FR)

(21) Appl. No.: 13/148,378

(22) PCT Filed: Feb. 12, 2010
(86) PCT No.: PCT/FR2010/000119

§ 371 Date: Aug. 8, 2011

(30) Foreign Application Priority Data
Feb. 13, 2009 (FR) 09 50954

(51) Int. Cl. B08B 1/00; B05D 3/00; A61K 8/00; B05D 7/00

(52) U.S. Cl. 15/104.94; 427/372.2; 510/130

(57) ABSTRACT

A skin-cleansing article that is activated by moistening with water, followed by a mechanical action so as to obtain a cleansing foam, includes a fibrous substrate and a cleansing composition. The substrate is a pad based on water-jet-entangled cotton fibres with a basis weight of between 100 and 300 g/m², and the cleansing composition includes at least one surfactant and a humectant such as glycerol, the amount of water on the substrate being less than 25% by weight of the article, and the article having from 0.1 to 1.2 gram of active material of the composition per gram of the substrate.

### BACKGROUND OF THE INVENTION

The present invention relates to a skin-cleansing article, which is disposable after use. The article comprises a pad made of a fibrous material forming a substrate, and a lotion with a substantially dry feel on the substrate.

In the field of cosmetics or for baby care, a fibrous pad based on cotton optionally mixed with other synthetic or artificial fibres, onto which is deposited a suitable cleansing or makeup-removing lotion or emulsion, depending on the case, is generally used for cleansing the skin.

Articles preimpregnated with lotion or emulsion, which can be used directly without needing to add additional cleansing or makeup-removing product at the time of its use, are now commercially available.

### BRIEF DESCRIPTION OF THE INVENTION

An embodiment of the invention relates to articles of the above-noted type, but where the lotion has a substantially dry appearance and feel on the substrate, i.e. the article contains overall a small amount of water, less than 25%. However, the article is not used in this form. To use the article, the lotion is activated by moistening it with water. The lotion contains agents via which a cleansing foam is formed when the article is subjected to a mechanical action, such as bending or rubbing. The pad impregnated with moist foam is then applied to the areas of skin that it is desired to cleanse.

The pads forming the substrate, which are also referred to as formats, are available in many forms. An embodiment of the invention is directed towards pads obtained from a lap of fibrous material made only of cotton or mixed with other fibres and in which they are chopped. Generally, the fibres of the lap are entangled so as to form a nonwoven arrangement of greater or lesser mechanical strength depending on the strength requirements of the article. The shape may be circular, oval, polygonal or some other shape, and the sizes may range from 25 to more than 100 cm². The basis weights are generally between 100 and 300 g/m².

A fibrous pad intended for such an application is chosen so as to have several characteristics. It is thick enough to remain comfortably in the hand when it is moistened. It absorbs and wipes away impurities. It conserves its integrity during use, on rubbing. It does not form fluff and does not leave fibres on the skin.

It is observed that, compared with a simple lap of carded cotton from which they were originally made, the mechanical properties of the pads have been improved in recent years by using one or other of the following two techniques:
incorporation into the mass of fibres of a meltable binder (in the form of fibres or powder), with heating with hot air or hot calendering; the binder agglomerates the cotton fibres when it is melted and then cooled, and affords an increase in strength of the pads in the three dimensions. However, this technique is not applicable to products that are intended to be made only of cellulose fibres;
treatment of the lap of fibres via mechanical means, preferably using water jets in a hydroentangling process, which interlace the fibres in the bulk and at the surface.

An embodiment of the invention relates to a fibrous substrate preferably obtained according to the above-noted second technique.

The hydroentangling process makes it possible to reduce the surface pilling capacity and to increase the tensile strength of the lap. This process, which is purely mechanical, allows the manufacture of laps composed up to 100% of cotton fibres.

The problem that must be confronted with a fibrous substrate whose fibres are mechanically entangled is that of avoiding substantial reduction of the mechanical strength of the pad after it has been moistened and its disintegration under the effects of the stretching forces to which it is subjected during use. Specifically, by virtue of some of the agents it comprises, a cleansing composition in principle shows greater slidability between the moistened fibres.

Loss of strength is not desired since it is sought to avoid the disintegration and destruction of the pad when the user rubs the skin or any other surface.

The Applicant set itself an advantage of producing a skin-cleansing article with a cleansing composition that is activated by moistening with water followed by a mechanical action, and incorporated into a fibrous substrate whose fibres are mechanically entangled, which, when it is moistened, conserves its pre-moistened strength properties or does not suffer a substantial reduction thereof, or even sees these properties improved.

The Applicant also set itself as an advantage an article that has a wet surface strength, after moistening, that is sufficient to limit the formation of pills on rubbing.

The intended advantages are achieved in accordance with an embodiment of the invention with an article characterized in that, the substrate being a pad based on water-jet-entangled cotton fibres with a basis weight of between 100 and 300 g/m², the cleansing composition comprises at least one surfactant, preferably a mixture of surfactants chosen from nonionic, anionic and amphoteric surfactants, and a humectant such as glycerol, the amount of water on the substrate being less than 25% by weight of the article, and the article comprising from 0.1 to 1.2 gram of active material of the composition per gram of substrate.

The active materials of the composition are all the elements except for water.

The substrate has a basis weight of between 150 and 250 g/m² and more particularly between 160 and 200 g/m².

In accordance with one characteristic, the article comprises from 0.2 to 0.72 gram of active material of the composition per gram of substrate. The composition comprises from 15% to 35% of the surfactant or surfactant mixture expressed as a percentage of active material of the composition and from 55% to 75% of the humectant expressed as a percentage of active material of the composition.

In accordance with another characteristic, the machine-direction strength of the article between its state before moistening and its wet state, after moistening, undergoes an increase of at least 10%.

In accordance with another characteristic, the composition comprises a gelling agent, but in small amount. The amount of gelling agent active material is less than 0.12%.

Advantageously, the composition comprises at least one additive such as a preserving agent, a dye, a fragrance, an acidity regulator, a skin-treating agent, etc.

According to one particular embodiment, the substrate is made 100% of cotton fibres. The substrate may, however, contain a certain percentage, from 5% to 30% and more particularly from 15% to 20%, of other natural fibres or synthetic or artificial fibres, in replacement for the cotton fibres.

The substrate may be obtained from a lap of fibres such as bleached cotton, formed pneumatically, by carding or by a combination of these techniques.

The layers are, for example, card webs. In the latter case, one embodiment consists in folding on itself, by means of an expanding-lapping machine, a web with a basis weight of between 30 and 60 g/m² at a lapping angle of between 0 and 90°. Several layers are thus superposed until the desired basis weight is obtained.

The substrate may also be formed from a plurality of layers of different nature. For example, the substrate may be formed from a lap obtained by pneumatic deposition of fibres between two or more webs.

According to another embodiment, the substrate comprises on one face a means forming a skin exfoliant. Advantageously, it is a pad manufactured according to the technique disclosed in patent application WO 2005/01699, which relates to the incorporation, immediately under the surface of the pad, of scrubbing elements. Thus, in accordance with one embodiment, the substrate comprises from 5 to 50 g/m² of scrubbing elements. These are natural organic elements such as strawberry achenes, apricot kernels, organic silica from bamboo or marrow cellulose, mineral elements such as silica beads, artificial elements such as cellulose and methylcellulose spheres, or synthetic elements such as polyethylene, Nylon, polypropylene or EVA polymers.

According to another embodiment of a lap with an exfoliant means, relatively rigid fibres of larger diameter are mixed with the cotton fibres. This mixture is carded to form a web that constitutes the surface web of the lap to be obtained. The rigid fibres are chosen from hemp, flax, sisal and yucca fibres. Depending on the efficacy of the exfoliant and the nature of the fibre, from 25% to 75% of these more rigid fibres are incorporated into the mixture in the surface web.

An embodiment of the invention also relates to a manufacturing process comprising a step of applying to a dry fibrous substrate a lotion having the cleansing composition containing from 25% to 45% and alternatively from 30% to 40% water. According to this process, an article that has a substantially dry feel, which does not need to be dried in order to be conditioned, is obtained after application of the lotion.

According to one variant, the process comprises a step of applying to a dry fibrous substrate an aqueous solution containing from 20% to 50% active material of the composition, followed by a drying step. This process allows application of the lotion into the bulk or only at the surface, but requires a drying step.

### BRIEF DESCRIPTION OF THE DRAWINGS

Other characteristics and advantages of the invention will emerge in greater detail in the description that follows and in which reference is made to the figures:

FIG. 1 is an illustration of a photograph taken with an optical microscope, at a magnification of 16×, of an outer face of a control sample N1 that has undergone the pilling test,

FIG. 2 is an illustration of a photograph taken with an optical microscope, at a magnification of 16×, of an outer face of a sample N2 in accordance with an embodiment of the invention, which has undergone the pilling test,

FIG. 3 is an illustration of a photograph taken with an optical microscope, at a magnification of 16×, of an outer face of a sample N3 in accordance with an embodiment of the invention, which has undergone the pilling test.

### DETAILED DESCRIPTION OF THE INVENTION

In an embodiment, the substrate is a hydroentangled pad having the following characteristics:
fibres: 100% bleached cotton;
basis weight: 180 grams per m²;
hydroentanglement: the two faces were subjected to a treatment with a plurality of water jets, spaced 2.5 mm apart and supplying an energy of 5 to 7×10³ kWh/m² on one face, and spaced 0.6 mm apart and supplying an energy of 2.08×10³ kWh/m² on the other face.

More generally, in the case of a lap of 160 to 200 g/m², a hydroentangling method comprises the treatment of one face with a supply of energy between 4 and 8×10⁻³ kWh/m² and 1 and 3×10⁻³ kWh/m² on the other face.

A non-limiting method of manufacture of a fibrous lap for the production of a pad forming the substrate according to an embodiment of the invention is reported hereinbelow.

A laminated cotton lap is prepared according to the lap manufacturing technique mentioned in patent EP 0 681 621 B1.

This technique consists in successively producing and in superposing three layers of raw cotton:
a first layer produced with a card, for example of pell-mell type,
a second layer produced by pneumatic lapping using a machine of Rando type, the fibres of the layer being oriented obliquely relative to the lower and upper horizontal planes of the lap, and finally
a third layer produced with a card and similar to the first.

This lap composed of three superposed layers is then chemically treated in order to make it hydrophilic and white. According to the technique described in the above-noted patent, the lap thus formed is then entrained by a liquid-permeable conveyor belt to the various posts for continuous line treatment.

The lap is impregnated by gravitational pouring of a kiering solution containing sodium hydroxide, onto the lap, in the form of a liquid blade transversely relative to the direction of movement of the lap. By means of a suction slit located under the web, a sufficient negative pressure is created to allow at least part of the solution to pass through the lap. At the same time, the amount of liquor supplied to the lap is controlled by adjusting the vacuum created by the suction slit. The lap is introduced into a vaporizer heated to a temperature in the region of 100° C. in which it resides, while remaining continuous, for a given time that especially depends on the material throughput.

The lap is then rinsed. The kiering fluid is extracted by means of a second liquid blade and a vacuum slit associated with a moderate vacuum. The kiered hydrophilic lap is impregnated with a bleaching solution containing hydrogen peroxide, in the same manner as for the kiering treatment. The lap is then introduced into a vaporizer heated to a temperature of about 100° C. in order for the bleaching to be effective. The lap is then rinsed by means of a succession of liquid blades associated with suction slits.

This treatment of the lap affords adhesion between the layers of which it is formed and very good cohesion to the assembly. This technique makes it possible to manufacture laps with a basis weight of between 80 and 600 g/m².

The cohesion of the lap is improved by means of a rinsing treatment according to the technique described in patent EP 0 805 888 B1, which consists in combining rinsing of the lap with hydroentanglement via fine jets of high-pressure water, which gives the lap an entangled surface state, without a free end of fibres, and good mechanical strength.

The jets are produced by means of injectors as used in the technique of hydrodynamic entanglement of nonwovens. Each injector comprises, for example, an elongated chamber, closed over its length by means of a plate that is perforated, in one or more rows, with a large number of small-diameter holes, of about 80 to 200 μm. The chamber is fed with liquid under pressure that escapes via the orifices in the form of fine parallel jets of corresponding diameter.

The level of energy to be supplied depends on the thickness of the lap and on its basis weight. Consolidation of the lap enables it to be transformed into an absorbent pad or a makeup-removing pad by simple cutting-out and packaging.

This rinsing treatment applied to the three-layer laminated lap described above also has the advantage of not excessively reducing the thickness of the lap, while at the same time reinforcing the superficial layers.

It is possible to apply a differentiated rinsing treatment on the two faces. Differentiated treatments are described in patent EP 1 106 723 B1 or in the divisional patent application EP 1 167 605 A1, which are incorporated herein by reference.

In an embodiment, a treatment comprises the entanglement of a first face with a plurality of water jets spaced 2.5 mm apart, while supplying an energy of 5 to 7×10⁻³ kWh/m². The entanglement of the second face is achieved by means of a plurality of water jets spaced 0.6 mm apart, while supplying an energy of 2.08×10⁻³ kWh/m². After entanglement, the lap is dried and cut into pads having the desired format via suitable cutting means.

### Composition of the Lotion

In accordance with an embodiment of the invention, the lotion comprises at least the following components:
a humectant such as glycerol,
a surfactant such as a nonionic, anionic or amphoteric surfactant capable of forming a foam.

The optional additives are as follows:
preserving agent such as those permitted in cosmetics and forming the list published in the Official Journal by application of directive No. 76/768/EEC,
fragrance,
dye,
skin-treating agent,
gelling agent,
acidity regulator.

As humectant, i.e. a hygroscopic agent for retaining water and thus for improving the moisturization of the skin, besides glycerol, another polyol may be used, for instance propylene glycol, butylene glycol, sorbitol, pentylene glycol or hexylene glycol.

The nonionic surfactant is preferably chosen from alkyl polyglucosides, for instance decyl glucoside.

The anionic surfactant is preferably one or more of the agents having one of the following three functional groups:
sulfates such as alkyl sulfates, alkyl ether sulfates including sodium laureth sulfate, and disodium sulfosuccinates;
sulfonates such as alkyl taurates, alkyl isethionates, alkyl aryl sulfonates or olefin sulfonates;
carboxylates such as sarcosinates or acyl glutamates.

The amphoteric surfactant is preferably one or more of the following agents, according to the classification in the “Handbook of Surfactants” M. R. Porter, 2nd edition, Blackie A&P;
amino propionate derivatives such as alkyl aminopropionates, alkyl ampho polycarboxy propionates, ampho propionates, ampho carboxy propionates, alkyl iminodipropionates, amino alkanoates, beta-N-alkylalanines, alkyl amino propionates, alkyl iminodipropionates, imino dialkanoate propionates;
betaine derivatives, for example cocamidopropyl-betaine, alkyls such as alkyl betaines, alkyl bisbetaines, alkyl dimethyl betaines, alkyl amido betaines, alkyl amido propyl betaines, alkyl amido propyl dimethyl betaine, alkyl amido propyl dimethyl sulfobetaines and alkyl amido propyl hydroxy sultaines; sulfo amido betaines; sulfo betaines;
glycinates such as alkyl glycinates, alkyl amino carboxylic acids, alkyl amphomonoacetates, alkyl amphodiacetates, alkyl carboxy glycinates, alkyl ampho polycarboxy glycinates, alkyl imino diglycinates, alkyl polyaminocarboxylates, amino alkanoates, ampho glycinates, ampho carboxy glycinates, carboxy glycinates, alpha-N-alkyl amino acetic acids.

### Tests

Tests aimed at checking firstly the mechanical strength properties and secondly the surface friction resistance were performed.

Three articles were tested.

A sample of control cotton N1 as defined in the above implementation example, namely: 100% bleached cotton, 180 g/m², the two faces of the lap were subjected to a treatment with a plurality of water jets, spaced 2.5 mm apart and supplying an energy of 6.7×10⁻³ kWh/m² on one face, and spaced 0.6 mm apart and supplying an energy of 2.08×10⁻³ kWh/m² on the other face.

A pad impregnated with a lotion whose percentage composition is as follows:

glycerol: 43.27
water: 35.94
anionic surfactant—sodium laureth sulfate: 11.52
amphoteric surfactant: cocamidopropylbetaine: 1.92
fragrance: 1.8
nonionic surfactant—decyl glucoside: 2.26
preserving agent: 1.30
potassium sorbate: 0.29
citric acid: 0.18
seaweed extract: 1.50
dye: 0.02
total: 100

The active materials of the composition are all the elements except for water.

A sample N2 is prepared by depositing the above lotion onto the substrate at a rate of 0.56 gram of lotion per gram of substrate. Since the lotion is relatively viscous, the deposition is performed using a lip nozzle. The article obtained has a level of dryness sufficient to make it unnecessary to dry it.

A sample N3 is prepared with a lotion of the same composition as above, diluted fourfold in water. The deposition is performed by padding, followed by drying. The final degree of impregnation of active materials is the same as for N2.

### Tensile Strength Measurement Test

The EDANA test referenced 20.2-89 for qualifying the behaviour of nonwoven textiles subjected to tensile stresses was applied.

The tensile strength values were measured on samples having the dimensions specified in the test, to which was applied a longitudinal tensile force at a constant rate of increase.

The tests were performed firstly on samples before wetting, and secondly on samples that were moistened, after wetting. Before wetting, for N1, its residual moisture content is less than 8%, for example in the case of cotton, and for N2 and N3, its residual moisture content is less than 25% by weight of the article.

For the humidification, the sample was folded in two at the middle, the fold being perpendicular to the length of the sample. The region of the fold over one centimetre is soaked for one second in distilled water, without draining. The sample is unfolded for the measurement.

Fifty tests were performed for each of the three laps N1, N2 and N3, before wetting and after wetting, respectively. The mean, maximum and minimum values for the strength values on samples cut in the machine direction, on the one hand before wetting (RSMS), and on the other hand after wetting (RSMH) in newtons are summarized in the table below:

| | N1 | | N2 | | N3 | |
|---|---|---|---|---|---|---|
| | RSMS | RSMH | RSMS | RSMH | RSMS | RSMH |
| Mean | 27.82 | 20.49 | 14.76 | 18.57 | 11.46 | 24.61 |
| Maximum | 34.61 | 27.42 | 22.46 | 25.99 | 16.89 | 30.30 |
| Minimum | 23.59 | 15.29 | 8.67 | 12.06 | 8.08 | 20.31 |
| Standard | 2.58 | 2.91 | 3.44 | 2.32 | 1.92 | 2.54 |
| deviation | | | | | | |

Comparison of the values obtained before wetting and after wetting gives the following variations for each sample relative to itself.

### N1: −26%

### N2: +26%

### N3: +115%

The strengths before wetting show that the impregnation causes strength to be lost, since the value of the strength of the lap impregnated in the bulk is lower than that of the surface-impregnated lap.

Surprisingly, after humidification, the strength characteristics of the pad containing the cleansing composition are improved, either at the surface or impregnated in the thickness, whereas an additional decrease was expected. A certain amount of strength is recovered, which goes beyond that of the control pad for the pad impregnated in the bulk.

### Pilling Test

According to this test, the laps are moistened by padding (the padding pressure is adjusted to the minimum 0 and the running speed is 0.7 m/minute). The degree of application is about 150%. They are then subjected to rubbing in accordance with the Martindale method, pilling version (NF EN ISO 12945-2):
without load (the mass of the specimen holder together with the stem is 155 g),
without felt under the sample,
replacing the wool web with the fabric used for the tests of fastness of dyes on rubbing according to standard NF EN ISO 105×12, and examining the specimens after 5 and 10 cycles.

The pills formed on the nonwoven are counted and measured by optical analysis.

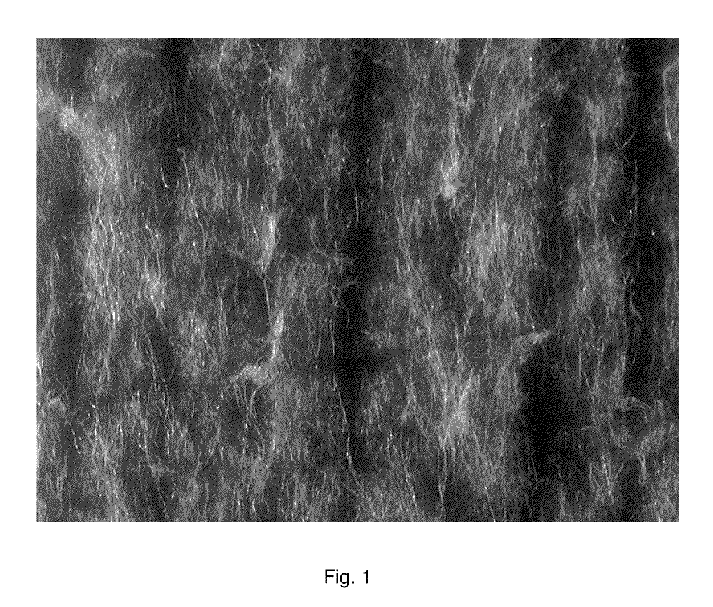

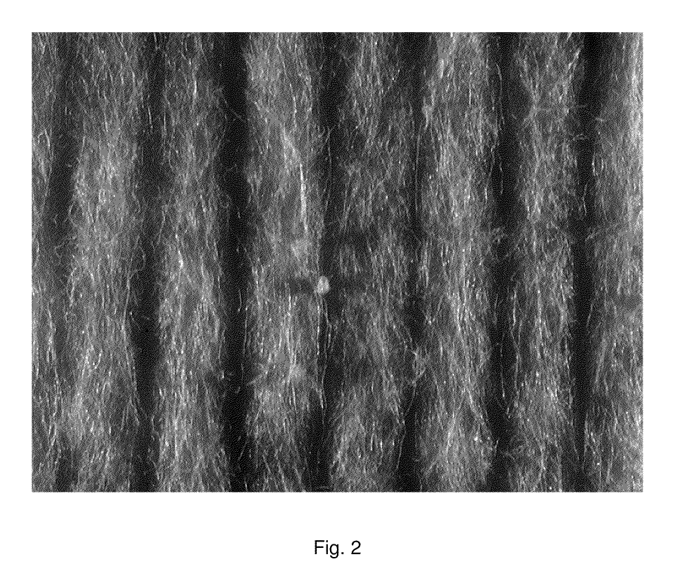

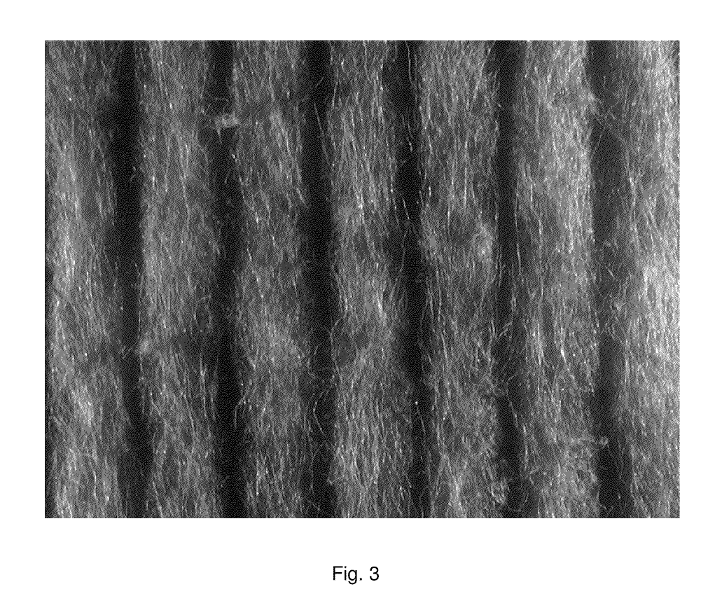

On a total area of 2084 mm², the number of pills and their cumulative area are measured. The samples subjected to the test were photographed, and the result is given in FIGS. 1 to 3 for, respectively, samples N1 to N3.

Test with 5 Cycles
N1: 116 pills with a total area of 21 mm²;
N2: 41 pills with a total area of 9 mm²;
N3: 43 pills with a total area of 6 mm²;
Test with 10 Cycles
N1: 178 pills with a total area of 34 mm²;
N2: 60 pills with a total area of 8 mm²;
N3: 69 pills with a total area of 9 mm².

These results highlight product N2, which shows not only a gain in strength during humidification, but also surface cohesion. Fewer pills are formed; and they are of smaller size.

1. A skin-cleansing article comprising:
a fibrous substrate and a cleansing composition that is activated by moistening with water followed by a mechanical action so as to obtain a cleansing foam, wherein:
the substrate is a pad comprising water-jet-entangled cotton fibres with a basis weight of between 100 and 300 g/m²,
the cleansing composition comprises
at least one surfactant, and
a humectant,
the amount of water on the substrate is less than 25% by weight of the article, and the article comprising from 0.1 to 1.2 gram of the cleansing composition per gram of the substrate.

2. The article according to claim 1, wherein the basis weight of the substrate is between 150 and 250 g/m².

3. The article according to claim 1, wherein the article comprises from 0.2 to 0.72 gram of the cleansing composition per gram of the substrate.

4. The article according to claim 1, wherein the cleansing composition comprises a mixture of surfactants chosen from nonionic, anionic and amphoteric surfactants.

5. The article according to claim 1, wherein a strength of the article, in a machine direction, between its state before moistening and its wet state, state after moistening, undergoes an increase of at least 10%.

6. The article according to claim 1, wherein the cleansing composition comprises, as an active material, from 15% to 35% of the at least one surfactant and from 55% to 75% of the humectant.

7. The article according to claim 1, wherein the cleansing composition further comprises, as an active material, a gelling agent in an amount of less than 0.2%.

8. The article according to claim 1, wherein the cleansing composition further comprises an additive in the form of a preserving agent, a dye, a fragrance or an acidity regulator.

9. The article according to claim 1, wherein the substrate comprises, on at least one face, a skin exfoliant.

10. The article according to the preceding claim 9, wherein the substrate comprises from 5 to 50 g/m² of scrubbing elements forming the skin exfoliant.

11. The article according to claim 1, wherein a number of pills, after rubbing according to a method defined as the Martindale method, pilling version (NF EN ISO 12945-2), is at least halved relative to the substrate without a lotion.

12. A process for manufacturing an article according to claim 1, comprising:
applying to a dry state of the fibrous substrate a lotion having a composition comprising at least one surfactant, and a humectant, and containing from 25% to 45% water.

13. A process for manufacturing an article according to claim 1, comprising:
applying to a dry state of the fibrous substrate an aqueous solution containing from 20% to 50% of the cleansing composition of claim 1, followed by drying the article.

14. The article according to claim 1, wherein the humectant is glycerol.

15. The article according to claim 1, wherein the basis weight of the substrate is between 160 and 200 g/m².

16. The article according to claim 1, wherein the cleansing composition further comprises, as an active material, a gelling agent in an amount of less than 0.12%.

17. The process according to claim 12, wherein the lotion contains from 30% to 40% water.
